(19) United States
(12) Patent Application Publication
(10) Pub. No.: US 2006/0142295 A1
(43) Pub. Date: Jun. 29, 2006

(54) Method of treating cancer with quinolone carboxylic acid derivatives

(76) Inventors: Jacques Dumas, Bethany, CT (US); Uday Khire, Hamden, CT (US); Shirley Lasch, Bethany, CT (US); Dhanapalan Nagarathnam, Bethany, CT (US); William J. Scott, Guilford, CT (US)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/546,091

(22) PCT Filed: Mar. 5, 2004
(86) PCT No.: PCT/US04/07267

§ 371 Date: Aug. 19, 2005

Related U.S. Application Data
(60) Provisional application No. 60/452,770, filed on Mar. 7, 2003.

(51) Int. Cl. A61K 31/506; A61K 31/496; A61K 31/497

(52) U.S. Cl. 514/252.11; 514/253.04; 514/253.08; 514/252.18

(57) ABSTRACT

This invention relates to a method of treating a hyper-proliferative disorder comprising the administration of an effective amount of a compound of Formula (I) to a patient in need thereof.

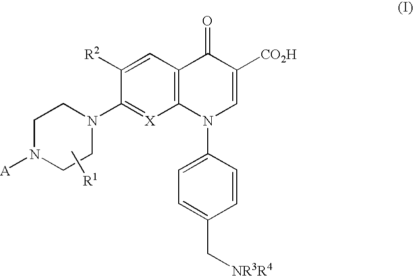

This patent application claims priority to U.S. Provisional Patent Application No. 60/452,770, filed Mar. 7, 2003.

### FIELD OF THE INVENTION

This invention relates to a method of using certain quinolone carboxylic acid derivatives for preventing or treating hyper-proliferative disorders.

### DESCRIPTION OF THE INVENTION

The present invention relates to a method of using the compounds and/or compositions described herein for treating or preventing, or, in the manufacture of a medicament for treating or preventing, mammalian hyper-proliferative disorders.

Accordingly, one embodiment of this invention is a method of treating a mammalian hyper-proliferative disorder comprising the administration to a patient in need thereof of an effective amount of a compound of Formula I 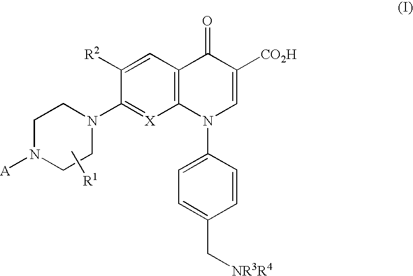
wherein
X represents a N atom or a group of the formula C—H, C—F or C—Cl;
A represents phenyl, naphthyl, pyridyl, pyrimidyl or pyrazinyl, each of which is optionally substituted with one, two or three substituents each independently selected from NO₂, CF₃, CN, OH, halo, (C₁-C₈)alkyl, (C₁-C₈)alkoxy, (C₁-C₈)acyl and (C₁-C₈)alkythio;
R¹ represents H or (C₁-C₆)alkyl;
R² represents H or halo;
R³ and R⁴ are each independently selected from H, benzyloxycarbonyl, (C₁-C₈)alkyl, (C₁-C₈)acyl, and
R³ and R⁴ together with the nitrogen atom to which they are attached form a 6-membered saturated heterocycle which additionally can optionally contain a further heteroatom selected from N, S or O;
and a pharmaceutically acceptable salt or ester thereof.

Another embodiment of this invention is a method of using the compounds of Formula I as prophylactic or chemopreventive agents for prevention of the mammalian hyper-proliferative disorders described herein. This method comprises administering to a patient in need thereof, including a human, an amount of a compound of Formula I, as described further herein, or a pharmaceutically acceptable salt or ester thereof, which is effective to delay or diminish the onset of the disorder.

The terms identified above have the following meaning throughout:

The term “optionally substituted” means that the moiety so modified may have from none to up to about the highest number of substituents indicated. When there are two or more substituents on any moiety, each substituent is defined independently of any other substituent and can, accordingly, be the same or different.

The terms “(C₁-C₆)alkyl” and “(C₁-C₈)alkyl” mean linear or branched saturated carbon groups having from about 1 to about 6 or 8 C atoms respectively. Such groups include but are not limited to methyl, ethyl, n-propyl, isopropyl, n-butyl, isobutyl, sec-butyl, tert-butyl, and the like.

The terms “(C₁-C₈)alkoxy” means a linear or branched saturated carbon group having from about 1 to about 8 C atoms, said carbon group being attached to an O atom. The O atom is the point of attachment of the alkoxy substituent. Such groups include but are not limited to methoxy, ethoxy, n-propoxy, isopropoxy, n-butoxy, isobutoxy, sec-butoxy, tert-butoxy, and the like.

The term “halo” means an atom selected from Cl, Br, F and I, where Cl, and F are preferred and F is most preferred.

The term “(C₁-C₈)alkylthio” means a linear or branched saturated carbon group having from about 1 to about 8 C atoms, said carbon group being attached to an S atom. The S atom is the point of attachment of the alkylthio substituent. Such groups include but are not limited to methythio, ethylthio, n-propylthio, isopropylthio, n-butylthio, isobutylthio, sec-butylthio, tert-butylthio, and the like.

The term “(C₁-C₈)acyl” means a linear or branched saturated carbon group having from about 1 to about 8 C atoms, said carbon group being attached to the core molecule through the C atom of a C═O group. Such groups include but are not limited to acetyl, propanoyl, heptaneoyl, hexanoyl, tert-butanoyl, iso-butanoyl, sec-butanoyl, and the like.

The term “a 6-membered saturated heterocycle which additionally can contain a further heteroatom selected from N, S or O” means a saturated ring containing one N atom and five other atoms, one of which is optionally another N, an S or an O atom, the rest being carbon. The “another N” or S or O atom may be located at any available location on the ring. Such groups include piperidine, piperazine, morpholine, and thiomorpholine. Preferred rings are those in which there is only one N atom, or there are 2 C atoms separating the N and the other heteroatom.

The compounds of Formula I that are preferred for use in this invention are those in which A is optionally substituted phenyl, pyridyl, pyrimidyl or pyrazinyl, and R³ and R⁴ are each independently H, (C₁-C₈)alkyl, and (C₁-C₈)acyl, and R³ and R⁴ together with the nitrigen atom to which they are attached form piperidine, piperazine, morpholine. Most preferred are those in which A is optionally substituted phenyl or pyridyl, R² is H, Cl or F, and R³ and R⁴ are each independently H or (C₁-C₆)alkyl.

Illustrative examples of the compounds of Formula I that can be used in the methods of treatment or prevention described herein include those compounds described in South African Patent Application No. 956013, filed Jul. 19, 1995, which is incoporated herein by reference. The compounds of Table I below are also illustrative of the compounds that may be used in this invention.

TABLE 1

| | |
|---|---|
| | (Ia) |
| 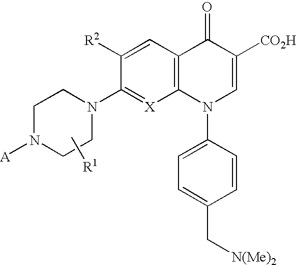 | |

| | | | | | MS |
|---|---|---|---|---|---|
| | | | | | m/z [M + H]+ |
| Example | | | | | HPLC |
| No. | A | X | R¹ | R² | (RT) |
| 1 | 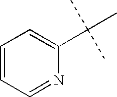 | CCl | H | F | 536 (1.75) |
| 2 | 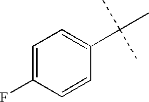 | CCl | H | F | 553 (2.53) |
| 3 | 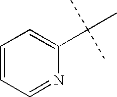 | CH | H | H | |
| 4 | 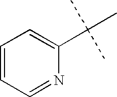 | CH | H | F | 502 (1.08) |
| 5 | 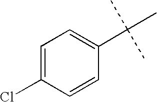 | CCl | H | F | 569 (2.82) |
| 6 | 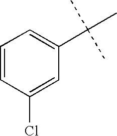 | CCl | H | F | 569 (2.82) |

The compounds of Table I correspond to the chemical names listed below, which were ascertained using the ACD/Lab Web service.

| Example | |
|---|---|
| No. | IUPAC Name |
| 1 | 8-chloro-1-{4-[(dimethylamino)methyl]phenyl}- |
| | 6-fluoro-4-oxo-7-[4-(2-pyridinyl)-1-piperazinyl]- |
| | 1,4-dihydro-3-quinolinecarboxylic acid |
| 2 | 8-chloro-1-{4-[(dimethylamino)methyl]phenyl}- |
| | 6-fluoro-7-[4-(4-fluorophenyl)-1-piperazinyl]-4- |
| | oxo-1,4-dihydro-3-quinolinecarboxylic acid |
| 3 | 1-{4-[(dimethylamino)methyl]phenyl}-4-oxo-7- |
| | [4-(2-pyridinyl)-1-piperazinyl]-1,4-dihydro-3- |
| | quinolinecarboxylic acid |
| 4 | 1-{4-[(dimethylamino)methyl]phenyl}-6-fluoro- |
| | 4-oxo-7-[4-(2-pyridinyl)-1-piperazinyl]-1,4-dihydro- |
| | 3-quinolinecarboxylic acid |
| 5 | 8-chloro-7-[4-(4-chlorophenyl)-1-piperazinyl]-1- |
| | {4-[(dimethylamino)methyl]phenyl}-6-fluoro-4- |
| | oxo-1,4-dihydro-3-quinolinecarboxylic acid |
| 6 | 8-chloro-7-[4-(3-chlorophenyl)-1-piperazinyl]-1- |
| | {4-[(dimethylamino)methyl]phenyl}-6-fluoro-4- |
| | oxo-1,4-dihydro-3-quinolinecarboxylic acid |

The use of a pharmaceutically acceptable salt of the compounds of this invention is also within the scope of this invention. The term “pharmaceutically acceptable salt” refers to either inorganic or organic salts of a compound of the present invention that have properties acceptable for the therapeutic use intended. For example, see: S. M. Berge, et al. “Pharmaceutical Salts,” J. Pharm. Sci. 1977, 66, 1-19.

Representative salts of the compounds of this invention also include the conventional non-toxic salts and the quaternary ammonium salts that are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate. The term acid addition salts also comprises the hydrates and the solvent addition forms which the compounds of this invention are able to form. Examples of such forms are, for example, hydrates, alcoholates and the like.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates including dimethyl, diethyl, and dibutyl sulfate; and diamyl sulfates, long chain halides such as decyl, lauryl, myristyl and strearyl chlorides, bromides and iodides, aralkyl halides including benzyl and phenethyl bromides, and others.

The esters of a compound of this invention are non-toxic, pharmaceutically acceptable esters such as alkyl esters including methyl, ethyl, propyl, isopropyl, butyl, isobutyl or pentyl esters. Additional esters such as phenyl-C₁-C₅ alkyl may be used, although methyl ester is preferred.

The compounds used in this invention may contain one or more asymmetric centers, depending upon the location and nature of the various substituents desired. Asymmetric carbon atoms may be present in the (R)- or (S)-configuration or may be mixtures of compounds with the (R)- and (S)-configurations. In certain instances, asymmetry may also be present due to restricted rotation about a given bond, for example, the central bond adjoining two substituted aromatic rings of the specified compounds. It is intended that all such configurations (including enantiomers and diastereomers) are included within the scope of the present invention. Preferred compounds are those with the absolute configuration of the compound of this invention which produces the more desirable biological activity. Separated, pure or partially purified isomers or racemic mixtures of the compounds of this invention are also included within the scope of the present invention.

Method of Making the Compounds of Formula I

In general, the compounds of the method of this invention may be prepared by standard techniques known in the art and by known processes analogous thereto. The compounds of Formula I can generally be synthesized, for example, according to the synthetic routes described in South African Patent Application No. 956013, filed Jul. 19, 1995, which is incorporated herein. It is believed that one skilled in the art, utilizing the preceding information, can utilize the present invention to its fullest extent. Nevertheless, the following are examples that can be used in preparing a compound of the method of the present invention. They are for illustrative purposes only, and are not to be construed as limiting the invention in any way.

Abbreviations and Acronyms

When the following abbreviations are used throughout this disclosure, they have the following meaning:
DABCO 1,4-Diazabicyclo[2.2.2]octane
Et ethyl
h hour(s)
HPLC high pressure liquid chromatography
LC-MS electropsray mass spectrometry
Me methyl
NMR nuclear magnetic spectroscopy
RT retention time

High pressure liquid chromatography-electrospray mass spectra (HPLC LC-MS) were obtained using either a:

(A) Hewlett-Packard 1100 HPLC equipped with a quaternary pump, a variable wavelength detector set at 254 nm, a YMC pro C-18 column (2×23 mm, 120 Å), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonitrile with 0.018% TFA. Gradient elution from 10% B to 95% over 3.5 minutes at a flow-rate of 1.0 mL/min was used with an initial hold of 0.5 minutes and a final hold at 95% B of 0.5 minutes. Total run time was 6.5 minutes.

or

(B) A Gilson HPLC system equipped with two Gilson 306 pumps, a Gilson 215 Autosampler, a Gilson diode array detector, a YMC Pro C18 column (2×23mm, 120 A), and a Micromass LCZ single quadrupole mass spectrometer with z-spray electrospray ionization. Spectra were scanned from 120-800 amu over 1.5 seconds. ELSD (Evaporative Light Scattering Detector) data was also acquired as an analog channel. The eluents were A: 2% acetonitrile in water with 0.02% TFA and B: 2% water in acetonitrile with 0.018% TFA. Gradient elution from 10% B to 90% over 3.5 minutes at a flowrate of 1.5 mL/min was used with an initial hold of 0.5 minutes and a final hold at 90% B of 0.5 minutes. Total run time was 4.8 minutes. An extra switching valve was used for column switching and regeneration.
Preparation of Compound Example 1 of Table 1

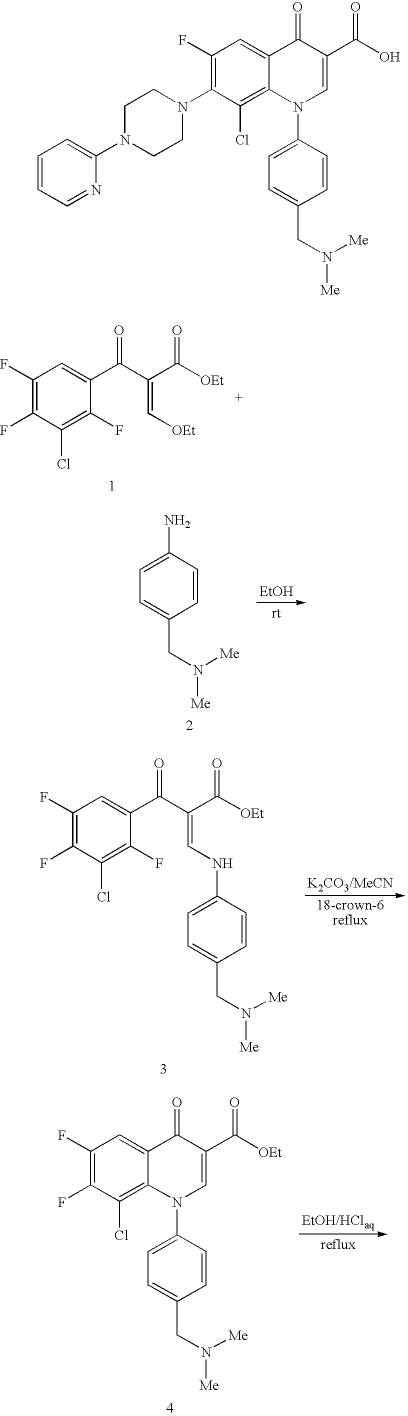

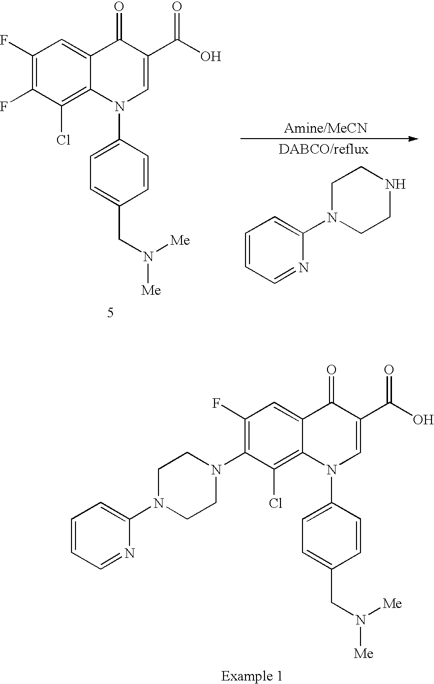

Starting material 1 (30.0 g, 89.0 mmol) was dissolved in ethanol (400 mL) and stirred at −10° C. Aniline 2 (14.7 g, 97.8 mmol) in ethanol (100 mL) was added slowly to the reaction solution. The reaction was allowed to warm up to room temperature and stirred for 3 h. The reaction mixture was evaporated to dryness and dissolved in dichloromethane. Crude intermediate 3 (44.0 g) was collected after passing through a short silica gel column using 50% ethyl acetate in hexane and 100% ethyl acetate, and removal of solvents in vacuo.

A solution of 3 (44.0 g, 99.8 mmol), potassium carbonate (27.6 g, 199.6 mmol), and 18-crown6 (7.9 g, 29.9 mmol) in acetonitrile (500 mL) was refluxed for 2 h, then cooled to room temperature, filtered, and concentrated. The intermediate 4 was purified by passing it through a short silica gel column and eluting it with methanol in dichloromethane (1% to 5 %). The pure intermediate 4 was dissolved in ethanol (200 mL) and 2N HCl (100 mL), then heated at 100° C. overnight. After removal of solvent, the yellow residue was washed with cold isopropanol, to yield the remaining white solid intermediate 5 (18 g, 45% overall yield). LCMS: 393 [M+1]⁺; RT=1.97 s. ¹H NMR (DMSO-d₆, in ppm): 8.64 (1H, s), 8.41 (1H, t), 7.82-7.76 (4H, m), 4.40 (2H, s), 2.75 (6H, s).

A solution of 5 (8 g, 20.4 mmol), 1-(2-pyridyl)piperazine (6.6 g, 40.7 mmol), DABCO (4.6 g, 40.7 mmol) in acetonitrile was heated at 100° C. for 3 days. The reaction was monitored by LCMS until the starting material 5 was consumed. The reaction was allowed to cool to room temperature, and the desired product precipitated as yellow solid. The solids were removed by filtration, washed with ethanol/ether (1/9), and purified by passing through a short silica gel column, eluting with 4% methanol in dichloromethane. After removal of the solvents using a vacuum pump, a light yellow solid (Example 1) was collected (4.3 g, 39% yield). LCMS: 536 [M+1]⁺; RT=1.75. ¹H NMR (CD₂Cl₂, in ppm): 8.75 (1H, s), 8.19-8.15 (2H, m), 7.55-7.45 (3H, m), 7.32 (2H, d), 6.65 (1H, d), 6.61 (1H, t), 3.65-3.59 (6H, br), 3.42 (4H, s), 2.30 (6H, s).

Other compounds of this invention, including those in Table I, can be made by processes analogous to the processes described in South African Patent Application No. 956013, filed Jul. 19, 1995, or by processes analogous to that described for Example 1 above, by substituting appropriate starting materials and/or other reagents, as would be readily recognized by one skilled in the art.

Generally, a desired salt of a compound of this invention can be prepared in situ during the final isolation and purification of a compound by means well known in the art. Or, a desired salt can be prepared by separately reacting the purified compound in its free base form with a suitable organic or inorganic acid and isolating the salt thus formed. For example, salts of the compounds identified herein can be made by isolating the compounds as hydrochloride salts, prepared by treatment of the free base with anhydrous HCl in a suitable solvent such as THF. Other compounds can be isolated as trifluoroacetic acid salts, which are formed during HPLC purification. These methods are conventional and would be readily apparent to one skilled in the art.

The compounds of the method of this invention may be esterified by a variety of conventional procedures including reacting the appropriate anhydride, carboxylic acid or acid chloride with the alcohol group of a compound of this invention. The appropriate anhydride is reacted with the alcohol in the presence of a base to facilitate acylation such as 1,8-bis[dimethylamino]naphthalene or N,N-dimethylaminopyridine. An appropriate carboxylic acid can be reacted with the alcohol in the presence of a dehydrating agent such as dicyclohexylcarbodiimide, 1-[3-dimethylaminopropyl]-3-ethylcarbodiimide or other water soluble dehydrating agents which are used to drive the reaction by the removal of water, and, optionally, an acylation catalyst. Esterification can also be effected using the appropriate carboxylic acid in the presence of trifluoroacetic anhydride and, optionally, pyridine, or in the presence of N,N-carbonyldiimidazole with pyridine. Reaction of an acid chloride with the alcohol can be carried out with an acylation catalyst such as 4-DMAP or pyridine.

One skilled in the art would readily know how to successfully carry out these as well as other methods of esterification of alcohols.

Additionally, sensitive or reactive groups on the compound of this invention may need to be protected and deprotected during any of the above methods. Protecting groups in general may be added and removed by conventional methods well known in the art (see, for example, T. W. Greene and P. G. M. Wuts, Protective Groups in Organic Synthesis; Wiley: New York, (1999).

Compositions Useful for the Method of This Invention

A compound of Formula I is useful in this method for preventing or treating the conditions described further herein when it is formulated as a pharmaceutically acceptable composition. A pharmaceutically acceptable composition is a compound of Formula I in admixture with a pharmaceutically acceptable carrier. A pharmaceutically acceptable carrier is any carrier that is relatively non-toxic and innocuous to a patient at concentrations consistent with effective activity of the active ingredient so that any side effects ascribable to the carrier do not vitiate the beneficial effects of the active ingredient.

Commonly used pharmaceutical ingredients which can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃);

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate);

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);

chelating agents (examples include but are not limited to edetate disodium and edetic acid);

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate);

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono-or polyvalent alcohols, saturated or unsaturated fatty alcohols, saturated or unsaturated fatty esters, saturated or unsaturated dicarboxylic acids, essential oils, phosphatdyl derivatives, cephalin, terpenes, amides, ethers, ketones and ureas);

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures);

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium; compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

The compounds of the present invention can be administered with pharmaceutically-acceptable carriers well known in the art using any effective conventional dosage unit forms formulated as immediate, slow or timed release preparations, including, for example, the following.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule which can be of the ordinary hard- or soft-shelled gelatn type containing, for example, surfactants, lubricants, and inert fillers such as lactose, sucrose, calcium phosphate, and corn starch.

A compound used in this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compounds of this invention may also be administered parenterally, that is, subcutaneously, intravenously, intraocularly, intrasynovially, intramuscularly, or interperitoneally, as injectable dosages of the compound in a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cafionic detergents, for example dimethyl dialkyl ammonium halides, alkyl pyddinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quaternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations which are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

It is believed that one skilled in the art, utilizing the preceding information, can utilize the present invention to its fullest extent. Nevertheless, the following are examples of pharmaceutical formulations that can be used in the method of the present invention. They are for illustrative purposes only, and are not to be construed as limiting the invention in any way.

Pharmaceutical compositions according to the present invention can be further illustrated as follows:
Sterile IV Solution: A 5 mg/mL solution of the desired compound of this invention is made using sterile, injectable water, and the pH is adjusted if necessary. The solution is diluted for administration to 1-2 mg/mL with sterile 5% dextrose and is administered as an IV infusion over 60 min.
Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 min.
Intramuscular suspension: The following solution or suspension can be prepared, for intramuscular injection:
   50 mg/mL of the desired, water-insoluble compound of this invention
   5 mg/mL sodium carboxymethylcellulose
   4 mg/mL TWEEN 80
   9 mg/mL sodium chloride
   9 mg/mL benzyl alcohol
Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.
Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.
Tablets: A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.
Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.
   Method of Treating Cancer

The compounds and compositions described herein can be used to treat or prevent hyper-proliferative disorders. An effective amount of a compound or composition of this invention can be administered to a patient in need thereof in order to achieve a desired pharmacological effect. A patient, for the purpose of this invention, is a mammal, including a human, in need of treatment (including prophylactic treatment) for a particular disorder described further herein. A pharmaceutically effective amount of compound or composition is that amount which produces a desired result or exerts an influence on the particular hyper-proliferative disorder being treated.

Hyper-proliferative disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

The disorders described above have been well characterized in humans, but also exist with a similar etiology in other mammals. Accordingly, the method of this invention can be administered to mammals, including humans, in need thereof for the treatment of angiogenesis and/or proliferative dependent disorders.

The anti-proliferative activity of the compounds of the method of the present invention can be illustrated, for example, by their activity in vitro in the in vitro tumor cell proliferation assay described below. The link between activity in tumor cell proliferation assays in vitro and anti-tumor activity in the clinical setting has been very well established in the art. For example, the therapeutic utility of taxol (Silvestrini et al. Stem Cells 1993, 11(6), 528-35), taxotere (Bissery et al. Anti Cancer Drugs 1995, 6(3), 339), and topoisomerase inhibitors (Edelman et al. Cancer Chemother. Pharmacol. 1996, 37(5), 385-93) was demonstrated with the use of in vitro tumor proliferation assays.

The compounds and compositions described herein, including salts and esters thereof, exhibit anti-proliferative activity and are thus useful to prevent or treat the disorders associated with hyper-proliferation. The following assay is one of the methods by which compound activity relating to treatment of the disorders identified herein can be determined.

In Vitro Tumor Cell Proliferation Assay

The adherent H460 human non-small cell lung carcinoma and Colo205 human colon carcinoma cell lines were purchased from the American Type and Culture Collection (ATCC, Manassas, Va.) and maintained in RPMI-1640 growth media supplemented with 10% heat inactivated fetal bovine serum (Gibco, Invitrogen Corp. Grand Island, N.Y.). At 37° C. in a humidified atmosphere of 5% CO₂.

The CellTiter 96® Aqᵤₑₒᵤₛ One Solution kit, MTS, (Promega, Madison, Wis.) was used to measure proliferation of tumor cell lines in vitro. This method monitors the bioreduction of a tetrazolium dye as a measure of cell viability. On Day 0, exponentially growing cells were trypsinized, resuspended in RPMI-1640 growth media supplemented with 10% FCS, 100 u/ml of penicillin G and 100 ug/ml of streptomycin sulfate, and seeded at 2000 cells per well into 96 well microtiter plates. Cells were incubated overnight in a humidified atmosphere of 5% CO₂ at 37° C. On Day 1, serial dilutions of compounds were prepared at 2× the finial assay concentration. One hundred microliters of 2× solution was added to test wells in duplicate and control wells received no test compound. The finial drug concentration ranged from 0 to 10-20 um in a 5 point dose-response curve. Cells were incubated in the presence of test compounds in a humidified atmosphere of 5% CO₂ at 37° C. for 72 hours. After 72 hours of compound exposure, 40 ul of Promega CellTiter 96® Aqₑₒᵤₛ One Solution was added to each well and absorbance at 490 nM was measured using a multi-well plate reader. Percent inhibition of proliferation was calculated using the following formula:
100×(1−Absorbance_{treated}−Background/(Absorbance_{control}−Background)

Where:

Absorbance_{treated}=absorbance at 490 nM in test wells, cells with test compound

Absorbance_{control}=absorbance at 490 nM in control wells, cells with no test compound

Background=absorbance 490 nM in wells containing media and no cells

The concentration of test compound required to inhibit proliferation of 50% of the cells (IC₅₀) was determined by linear regression analysis. Representative compounds described for use in the present invention were found to exhibit anti-proliferative activity in these assays.

Based upon the above and other standard laboratory techniques known to evaluate compounds useful for the prevention or treatment of the diseases or disorders described above by standard toxicity tests and by standard pharmacological assays for the determination of the prevention or treatment of the conditions identified above in mammals, and by comparison of these results with the results of known medicaments that are used to treat these conditions, the effective dosage of the compounds of this invention can readily be determined for prevention or treatment of each desired indication. The amount of the active ingredient to be administered in the prevention and/or treatment of one of these conditions can vary widely according to such considerations as the particular compound and dosage unit employed, the mode of administration, the duration of treatment (including prophylactic treatment), the age and sex of the patient treated, and the nature and extent of the condition to be prevented and/or treated.

The total amount of the active ingredient to be administered will generally range from about 0.001 mg/kg to about 300 mg/kg, and preferably from about 0.10 mg/kg to about 150 mg/kg body weight per day. A unit dosage may contain from about 0.5 mg to about 1500 mg of active ingredient, and can be administered one or more times per day. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of administration and number of doses of a compound or composition of the present invention or a pharmaceutically acceptable salt or ester thereof can be ascertained by those skilled in the art using conventional prevention and/or treatment tests.

The compounds of this invention can be administered as the sole pharmaceutical agent or in combination with one or more other pharmaceutical agents where the combination causes no unacceptable adverse effects. For example, the compounds of this invention can be combined with other anti-hyper-proliferative or other indication agents, and the like, as well as with admixtures and combinations thereof.

For example, optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment and/or prevention of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of this invention include but are not limited to other anti-cancer agents such as epothilone, irinotecan, raloxifen and topotecan.

It is believed that one skilled in the art, using the preceding information and information available in the art, can utilize the present invention to its fullest extent. It should be apparent to one of ordinary skill in the art that changes and modifications can be made to this invention without departing from the spirit or scope of the invention as it is set forth herein.

What is claimed is:

1. A method of treating a mammalian hyper-proliferative disorder comprising the administration to a patient in need thereof of an effective amount of a compound of Formula I 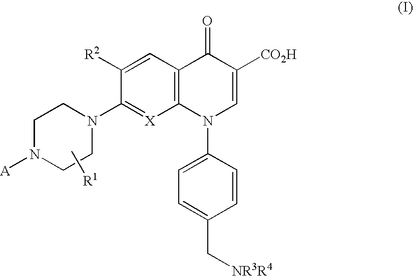
wherein
X represents a N atom or a group of the formula C—H, C—F or C—Cl;
A represents phenyl, naphthyl, pyridyl, pyrimidyl or pyrazinyl, each of which is optionally substituted with one, two or three substituents each independently selected from NO₂, CF₃, CN, OH, halo, (C₁-C₈)alkyl, (C₁-C₈)alkoxy, (C₁-C₈)acyl and (C₁-C₈)alkythio;
R¹ represents H or (C₁-C₆)alkyl;
R² represents H or halo;
R³ and R⁴ each independently represent H, benzyloxycarbonyl, (C₁-C₈)alkyl, or (C₁-C₈)acyl, or
R³ and R⁴ together with the nitrogen atom to which they are attached form a 6-membered saturated heterocycle which additionally can optionally contain a further heteroatom selected from N, S or O;
or a pharmaceutically acceptable salt or ester thereof.

2. A method according to claim 1 comprising the administration of a compound of Formula I wherein
X represents a group of the formula C—H, C—F or C—Cl
A represents phenyl, pyridyl, pyrimidyl or pyrazinyl each optionally substituted with one or two substituents each independently selected from NO₂, CF₃, CN, OH, halo, (C₁-C₈)alkyl, and (C₁-C₈)alkoxy;
R¹ represents H or (C₁-C₆)alkyl;
R² represents H or halo;
R³ and R⁴ each independently represent H, benzyloxycarbonyl, (C₁-C₈)alkyl, or (C₁-C₈)acyl;
or a pharmaceutically acceptable salt or ester thereof.

3. A method according to claim 1 comprising the administration of a compound of Formula I wherein
A represents pyridyl or phenyl, each optionally substituted with one or two halo atoms;
X represents C—H, C—F or C—Cl;
R¹ represents H;
R² represents H or halo; and
R³ and R⁴ each independently represent H, (C₁-C₈)alkyl, or (C₁-C₈)acyl;
or a pharmaceutically acceptable salt or ester thereof.

4. A method according to claim 1 wherein the mammalian hyper-proliferative disorder is selected from solid tumors, lymphomas, sarcomas and leukemias.

5. A method according to claim 4 wherein the disorder is selected from solid tumors.

6. A method according to claim 5 wherein the tumor is selected from cancers of the breast, reproductive organs, respiratory tract, brain, head, neck, hematopoietic tissue, digestive tract and urinary tract.

7. A method according to claim 6 wherein the disorder is selected from cancers of the breast, prostate, ovary, lung, colon, head, neck and hematopoietic tissue.
